(19) United States
(12) Patent Application Publication
(10) Pub. No.: US 2017/0156735 A1
(43) Pub. Date: Jun. 8, 2017

(54) INCISION COMPRESSION DEVICE

(71) Applicant: Steven Parker, Spokane, WA (US)

(72) Inventor: Steven Parker, Spokane, WA (US)

(21) Appl. No.: 14/960,330

(22) Filed: Dec. 4, 2015

(51) Int. Cl. A61B 17/132; A61F 5/34; A61F 5/32

(52) U.S. Cl. CPC A61B 17/1325; A61F 5/32; A61F 5/34

(57) ABSTRACT

A post-surgery compression device is disclosed herein to reduce the possibility of post-surgery infirmities. For instance, after the implantation of cardiac devices such as a pace maker or defibrillator, the compression device herein may adjustably attach to cover-up the incision area. The compression device includes a housing attached to a frame, the housing having a recess thereon. When the compression device is attached to a person, the recess is positioned over the post-surgical incision in order to reduce the amount of pressure on the incision itself, while simultaneously applying pressure to the area via the housing. The compression device reduces the possibility of post-surgery infirmities, including hematoma.

### BACKGROUND

Many people undergo cardiac surgery to receive an implant of a pacemaker, defibrillator or other similar such devices. These cardiac devices are inserted via a cutaneous incision near the heart of the person or patient. Bandages, glue, gauze pads, etc. are used to cover up the incision area post-surgery. Sometimes the cover-up materials result in post-surgery infirmities, including the possibility of hematoma.

### SUMMARY

A compression device that is capable of covering up post-surgical incisions while reducing the possibility of post-surgery infirmities is disclosed herein. The compression device applies pressure to the area surrounding the incision, while simultaneously applying little pressure to the surgical incision itself as a result of a recess strategically positioned on a bladder, balloon, or other housing type material on the compression device. The housing is attached to a frame which is secured to a body of a person via a strap, the strap being capable of adjusting the amount of pressure applied by the compression device and the strap also permitting conformity to the body type and size of a user.

A compression device is disclosed herein, the compression device including a frame including a first opening; a strap secured through the first opening, wherein the strap is configured to secure the compression device to a body of a user; and a housing that is attached to the frame, the housing having a bottom side and a top side, wherein the bottom side is configured to rest against a body of a user and the top side is opposite the bottom side, the bottom side of the housing having a recess that extends from a first end of the housing to a second end of the housing, wherein the first and second ends are opposite each other.

As another example, the housing is removably attachable to the frame. As a further example, multiple types of housings are attachable to the frame. As another example, the housing is removably attachable to the frame via a press fit or friction fit. IN another example, the housing includes multiple recesses thereon. As another example, the housing is filled with a liquid or a solid. In that example, the liquid includes a saline solution. As another example, the compression device includes a valve positioned on the top side of the housing, wherein the valve permits the removal and addition of gas or liquid into the housing. In a further example, the recess is off-centered, wherein the off-centered recess is configured to cover an incision or wound based on the positioning of the incision or wound on the body of the user.

A kit is disclosed herein, the kit includes a frame including a first opening; a strap secured through the first opening, wherein the strap is configured to secure compression device to a body of a user; and a first housing that is attachable to the frame, the first housing having a bottom side and a top side, wherein the bottom side is configured to rest against a body of a user and the top side is opposite the bottom side, the bottom side of the first housing having a first recess that extends from a first end of the first housing to a second end of the first housing, wherein the first and second ends are opposite each other, the first housing including a gas, liquid, or solid material therein.

The kit further includes a second housing having a second recess that is attachable to the frame, wherein the second housing includes a different gas, liquid, or solid material therein from the first housing. As another example, the first and second housings are removably attachable to the frame via a press fit or friction fit. In another example, the first housing includes multiple recesses thereon. As another example, the first housing stores a saline solution. In another example, the kit further includes a valve positioned on the top side of the first housing, wherein the valve permits the removal and addition of the gas or liquid into the housing. As another example, the recess is off-centered and the off-centered recess is configured to cover an incision or wound based on the positioning of the incision or wound on the body of the user.

### BRIEF DESCRIPTION OF THE DRAWINGS

FIG. 1 depicts a front view of a compression device in accordance with aspects of the disclosure.

FIG. 2 is a perspective view of a top portion of the compression device in accordance with aspects of the disclosure.

FIG. 3 is a side view of the compression device in accordance with aspects of the disclosure.

FIG. 4 is another side view of the compression device in accordance with aspects of the disclosure.

FIG. 5 is a perspective view of a bottom portion of the compression device in accordance with aspects of the disclosure.

FIG. 6 is a top view of the compression device in accordance with aspects of the disclosure.

FIG. 7 is a cross-sectional view of the compression device in accordance with aspects of the disclosure.

FIG. 8 depicts a material stored in the housing in accordance with aspects of the disclosure.

FIG. 9 is an example of a syringe that may be used with a valve of the compression device in accordance with aspects of the disclosure.

FIG. 10 is another example of the syringe that may be used with the compression device in accordance with aspects of the disclosure.

FIGS. 11A-C illustrate the compression device attached to a strap secured to a user in accordance with aspects of the disclosure.

FIGS. 12A-B depict alternative positions of the compression device in accordance with aspects of the disclosure.

FIGS. 13A-K are additional variations and embodiments of the compression device in accordance with aspects of the disclosure.

### DETAILED DESCRIPTION

The aspects, features and advantages of the present disclosure will be appreciated when considered with reference to the following description of preferred embodiments and accompanying figures. The following description does not limit the disclosure; rather, the scope is defined by the appended claims and equivalents. While certain processes in accordance with example embodiments are shown in the figures as occurring in a linear fashion, this is not a requirement unless expressly stated herein. Different processes may be performed in a different order or concurrently.

The present disclosure describes a compression device that reduces the possibility of hematoma in a person. The compression device may include a frame that is attached to a housing. The housing may hold materials therein such as a liquid, gas, or solid material therein. The compression device may also be attached to a strap or other mechanism that secures the compression device to the person. The housing may also include a recess on a side of the housing that comes in contact with the epidermis of the person. When the compression device is secured to the person, such as by using the strap, the recess portion of the housing is positioned over an incision of the person, such as an incision after a surgery, to assist in subcutaneous or sub-muscular vessel clot formation, thereby reducing the possibility of hematoma.

It should be noted that although the disclosure herein references using the compression device with human beings, the present disclosure is not limited thereto, but rather may be used with animals as well. In addition, although the disclosure herein may reference the use of the compression device post-surgery and with reference to the chest area, the present invention should not be limited thereto, rather, the compression device may be used at any location of a person or animal to assist in the development of blot clot formation, including the head, legs, arms, hands, abdomen, etc.

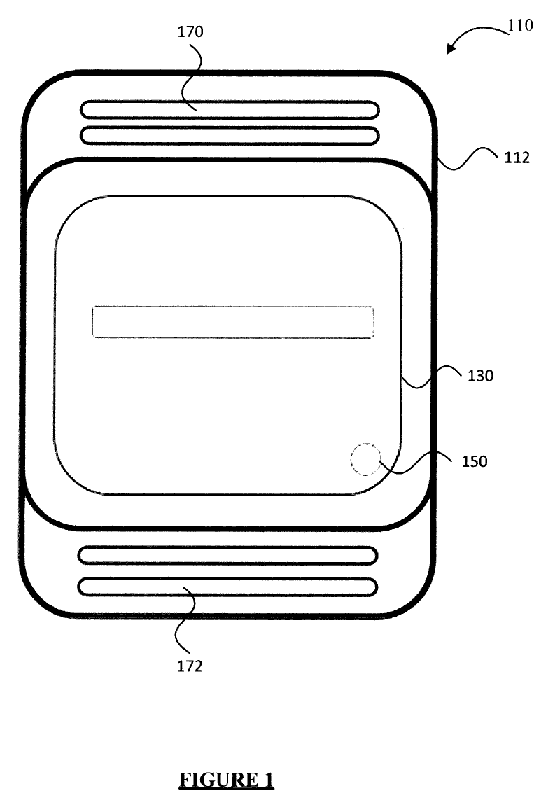

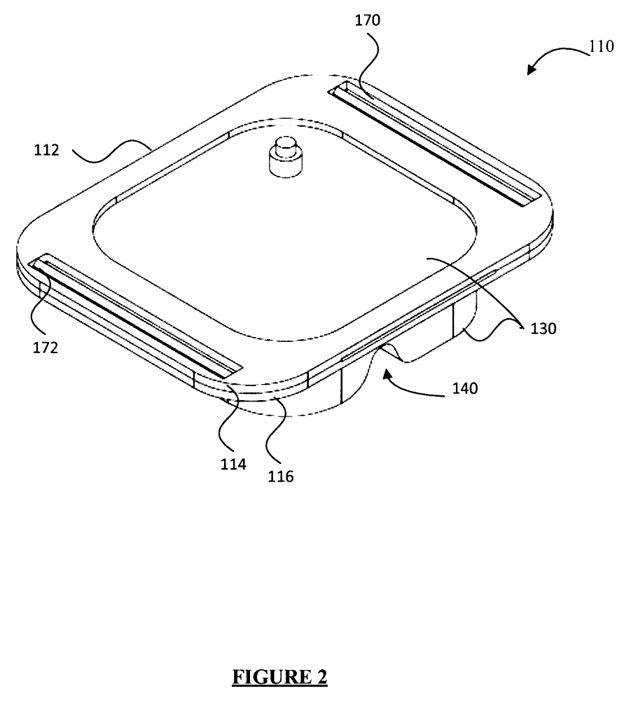

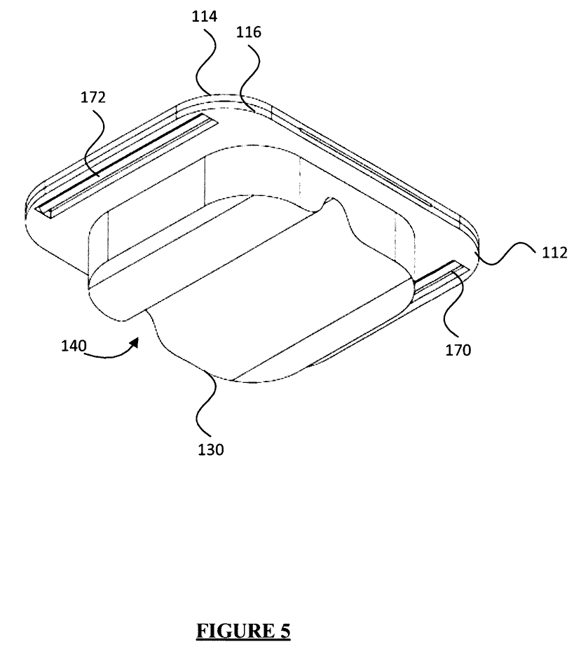

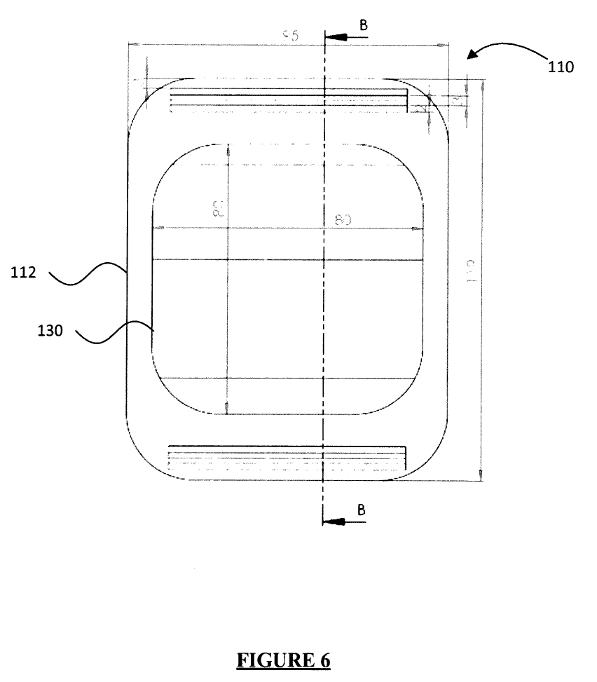

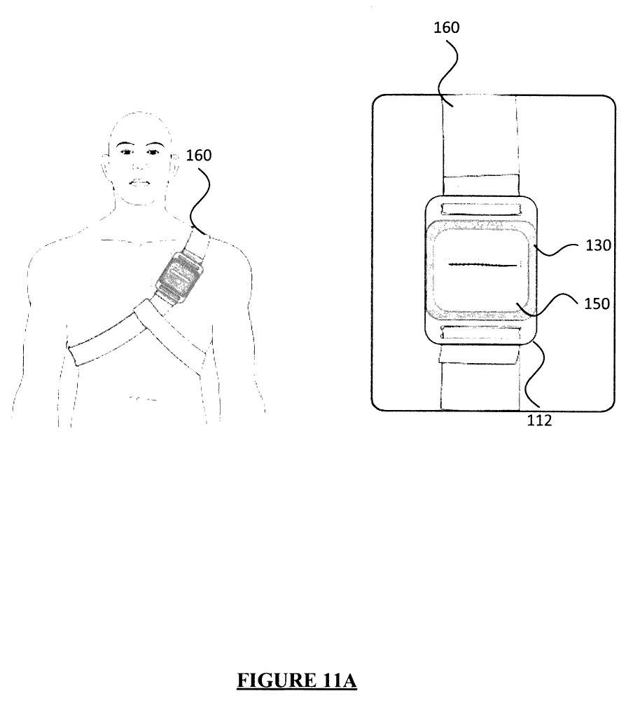

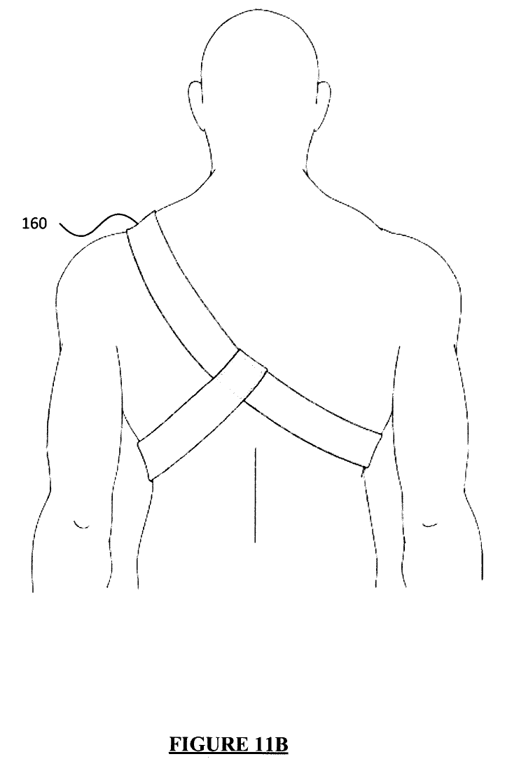

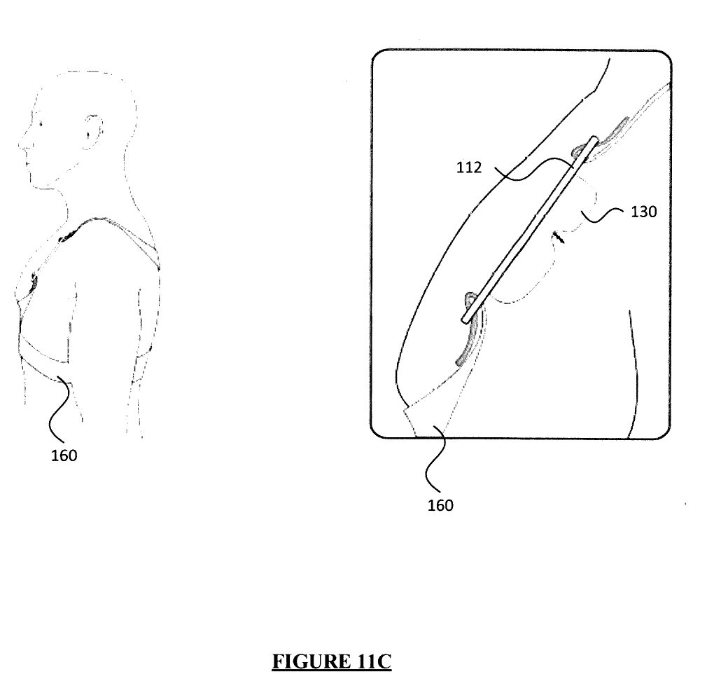

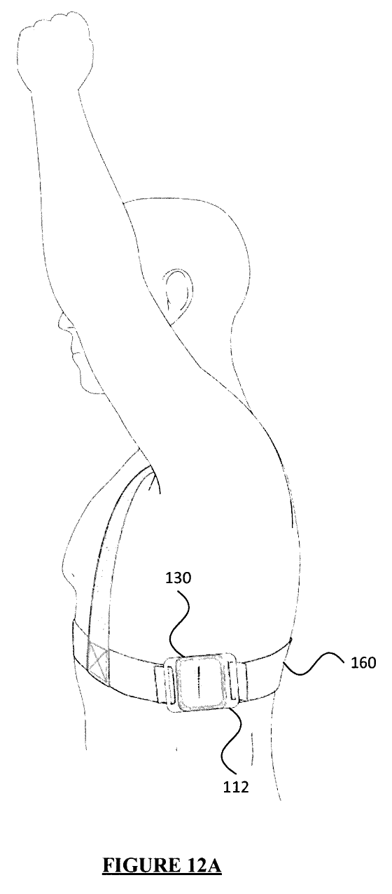

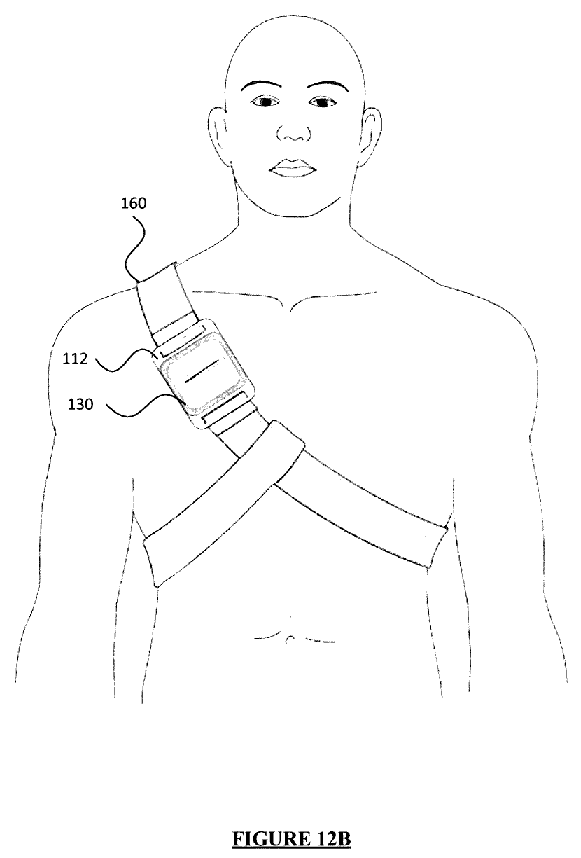

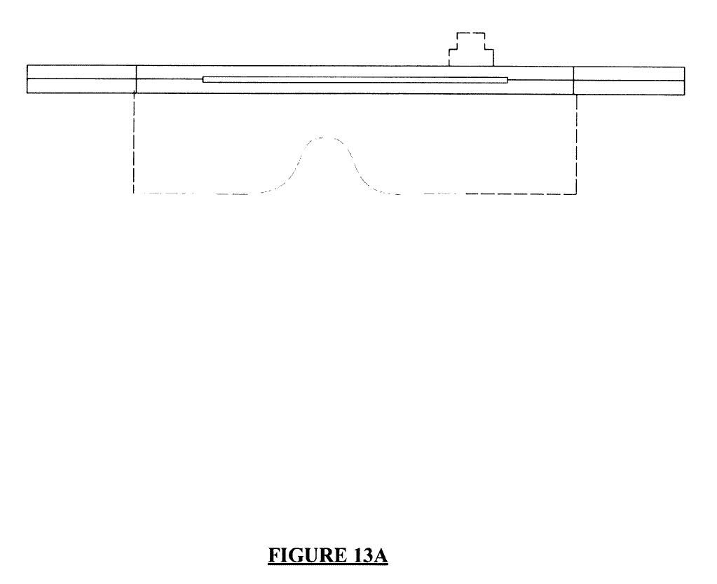

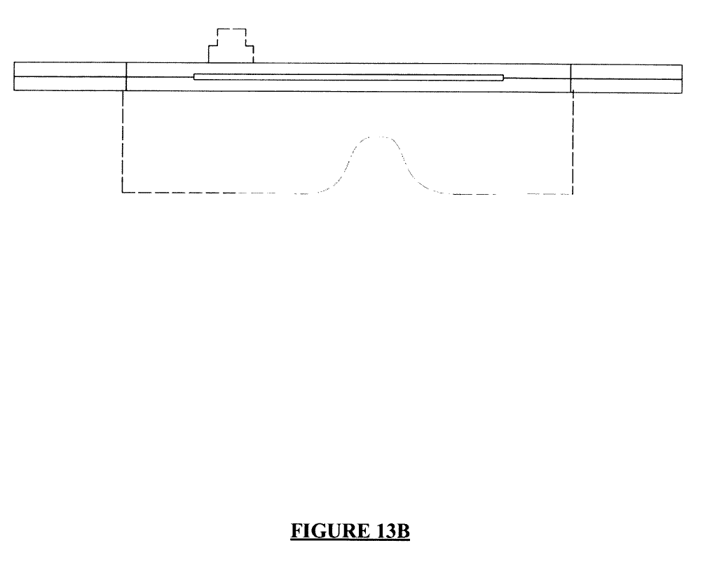

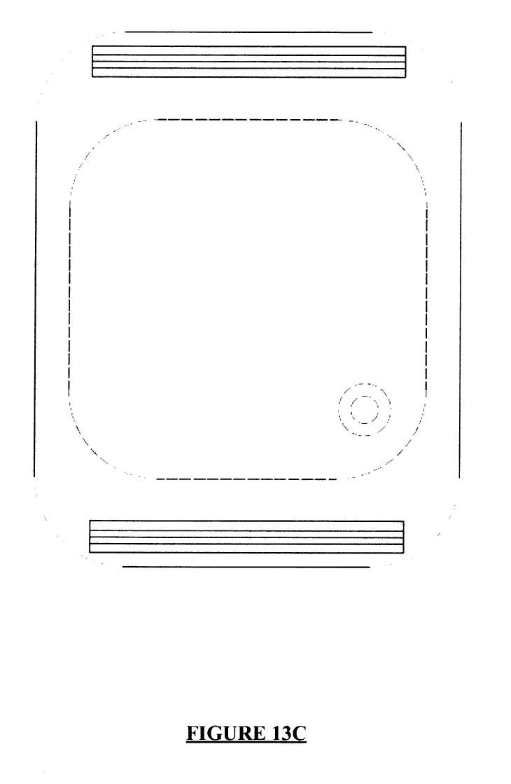

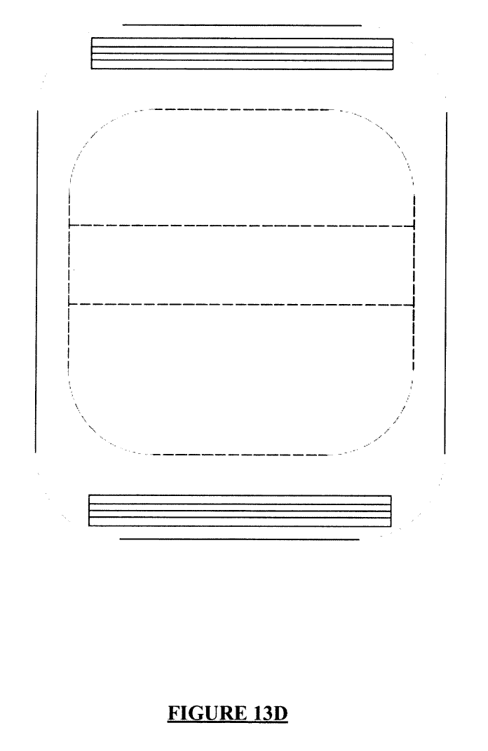

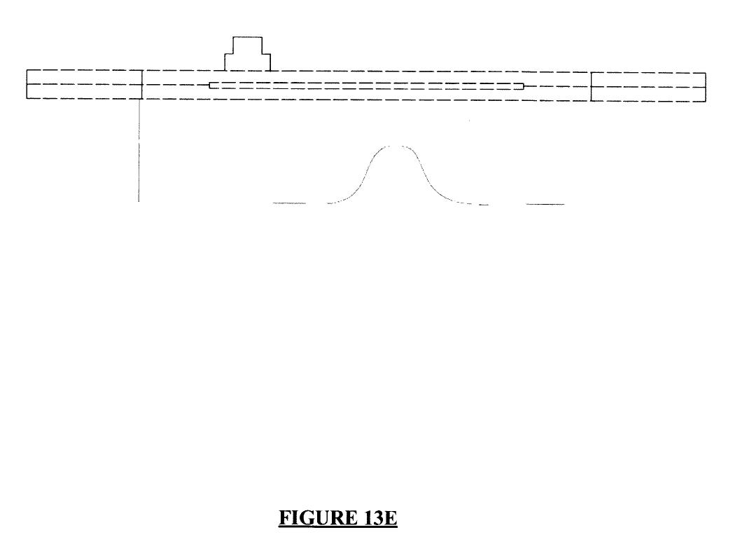

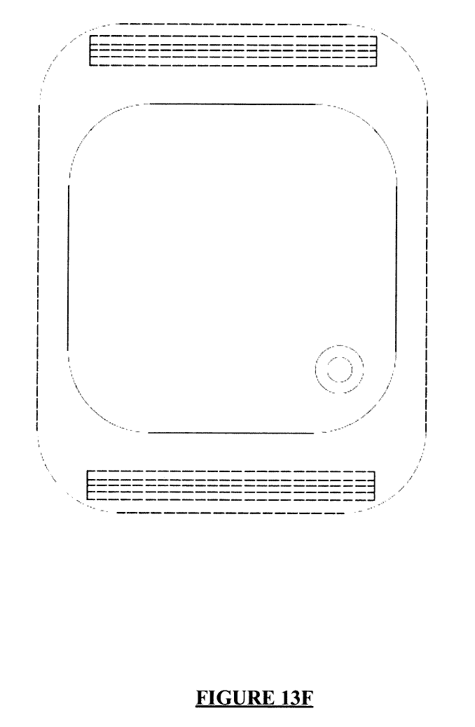

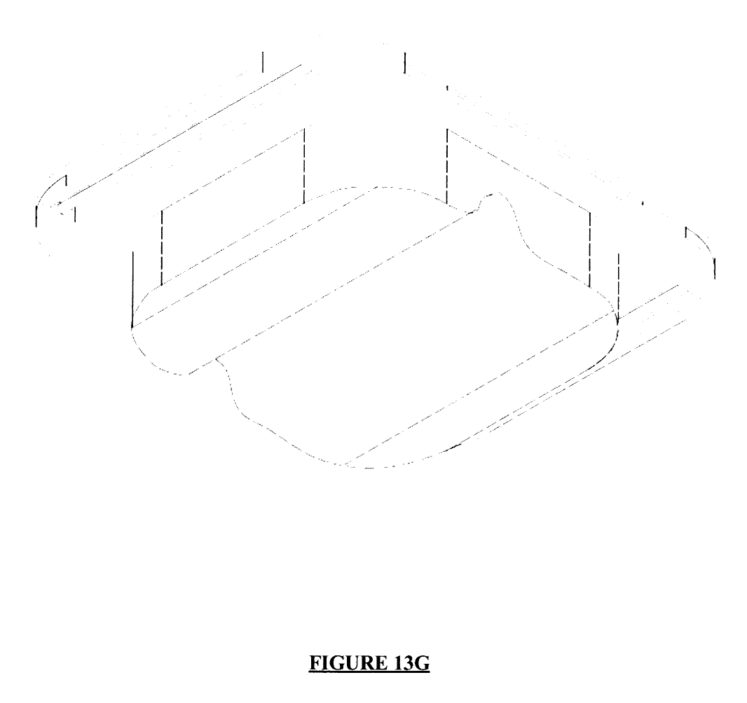

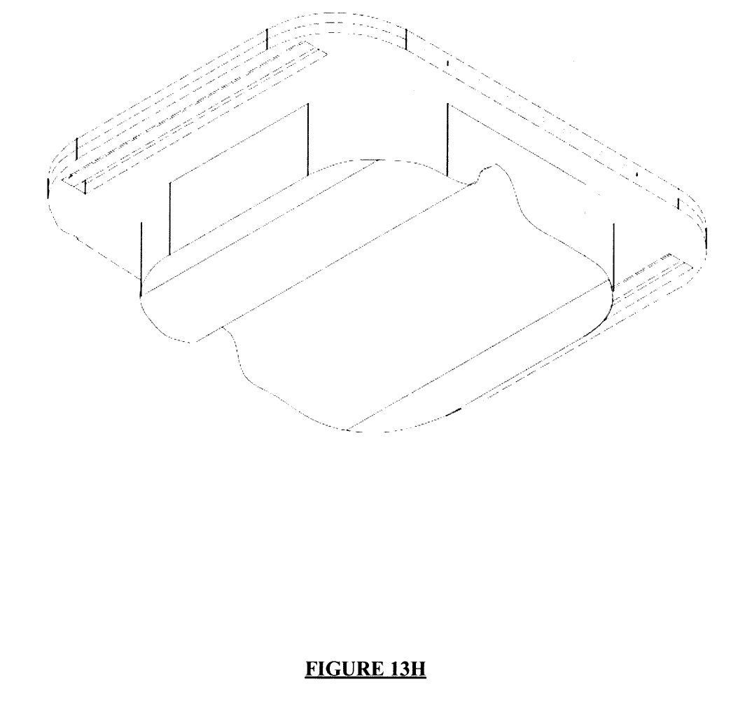

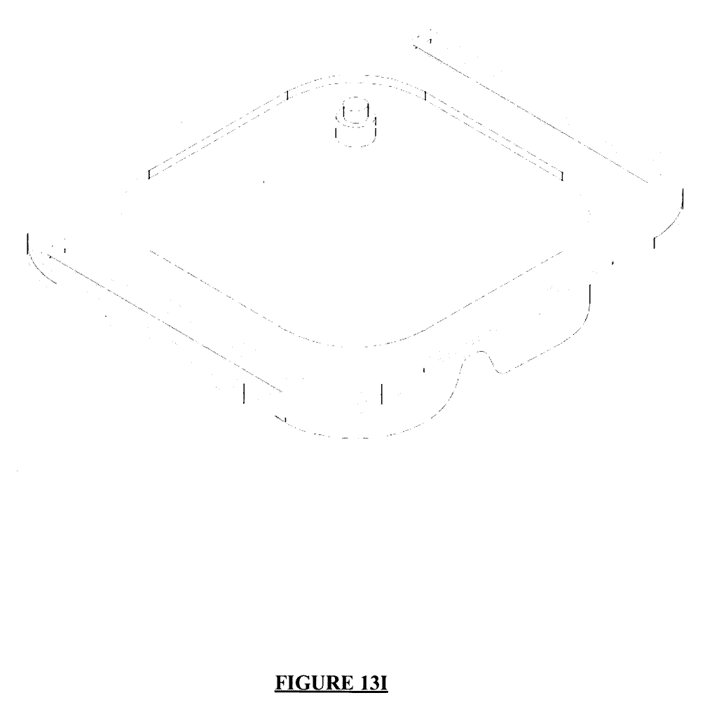

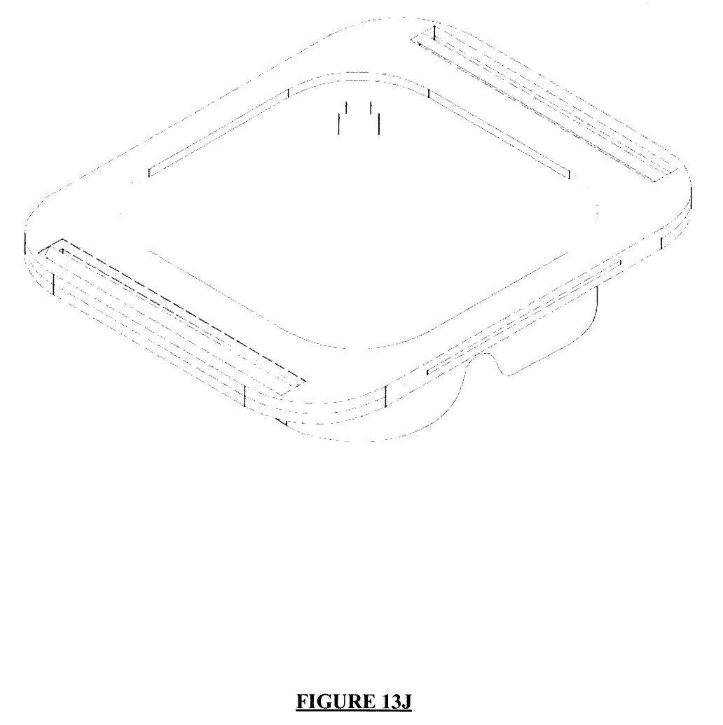

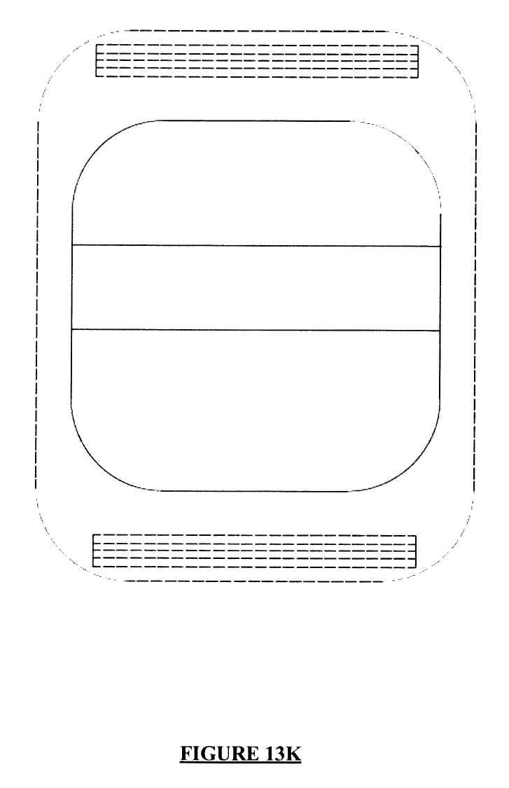

Referring to FIGS. 1-7, compression device 110 includes a frame 112 that is attached to housing 130. Frame 112 may be made of plastic, metal, or any material that is capable of maintaining its given shape. Housing 130 may be made of a flexible material that is capable of adjusting to the shape of a skin of the person, such as a rubber material, thin plastic, etc. Housing 130 may be empty or filled with a material such as gas, liquid, or a solid. FIG. 8 depicts housing 130 with a material stored therein, such as saline solution. In this regard, housing 130 may be compared to a bag or sack that is capable of holding materials therein while attached to frame 112. As an alternative, housing 130 may be referred to as a bladder or balloon because it is capable of housing various materials.

Housing 130 may be securely attached to frame 112. For example, as illustrated in FIGS. 1-7, frame 112 includes an upper portion 114 and a lower portion 116 that secures housing 130 in place. Specifically, housing 130 is positioned in between upper portion 114 and lower portion 116. Upper and lower portions 114 and 116 may lock into each other via a tab and notch mechanism, press-fit, friction fit, etc. As an alternative, housing 130 may be glued to frame 112. As another alternative, compression device 110 may include a tab and notch mechanism such that a tab in housing 130 may fit within a notch in frame 112. Housing 130 may be completely sealed such that any materials therein, such as gas or liquid, are incapable of escaping. It should be noted that housing 130 may not be sealed depending on the scenario, such as if housing does not include any liquid therein then there is no reason to completely seal housing 130.

Housing 130 may be removably attachable to frame 112. For example, housing 130 may be replaced with another, distinct housing. In this regard, upper and lower portions 114 and 116 of frame 112 may be separated to easily remove housing 130. At this point, a person may switch out the currently used housing 130 with another housing. A user may switch out the housings if, for example, the user wants a different housing that houses different materials or if the currently used housing is old, worn, or perhaps dirty after use.

For example, if a user is currently using a housing with gas, the user may switch the air housing with a liquid housing, such as a housing that contains saline solution. In this regard, a housing with saline solution may cause a cooling effect over the incision. Furthermore, a person may want to switch housings based on the particular time of day, such as if the person is active during the day or about to go to bed at night. Even further, the type of housing attached may change based on the particular healing stage the person is at. For instance, one housing may be more useful early after surgery, while another housing may be more useful longer after surgery.

As a further example, comfort of a particular housing may play a role in the person's decision on what housing to use, such as different materials or other characteristics about a particular housing. For example, the external layer of housing 130 that comes in contact with a person's skin may include lumps, projections of various sizes, or it may be smooth or anything in between. Alternatively, the material used may be more coarse or smooth depending on the housing used. For all of these reasons, a user may want to use different housings or a doctor prescribing the housing may change based on the particular situation.

A recess 140 may be positioned on housing 130 as well. In this regard, recess 140 is positioned over the incision or wound to reduce direct pressure thereon, while housing 130 itself is still able to apply pressure to the area directly around the incision or wound. Therefore, compression device 130 is able to apply pressure to the post-surgical site while simultaneously not putting too much pressure directly on the incision and wound as a result of the strategically placed recess.

Recess 140 may be of various sizes in various positions on housing 130. For example, depending on the size of an incision or other wound, a housing with a deeper or shallower depth may be better for the situation. Alternatively, if an incision or wound is positioned in an obscure location of the body, then recess 140 on the housing may be positioned off-centered on another location of the housing in order to be placed over the incision or wound.

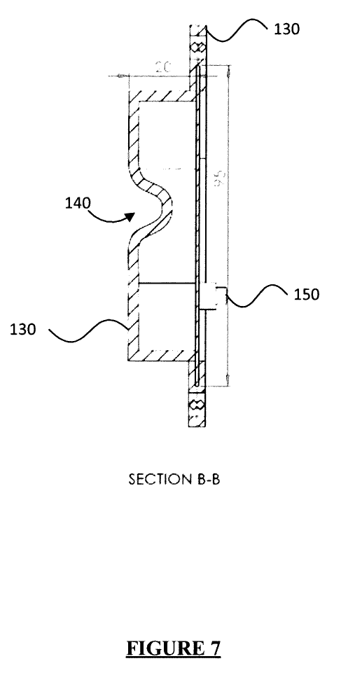

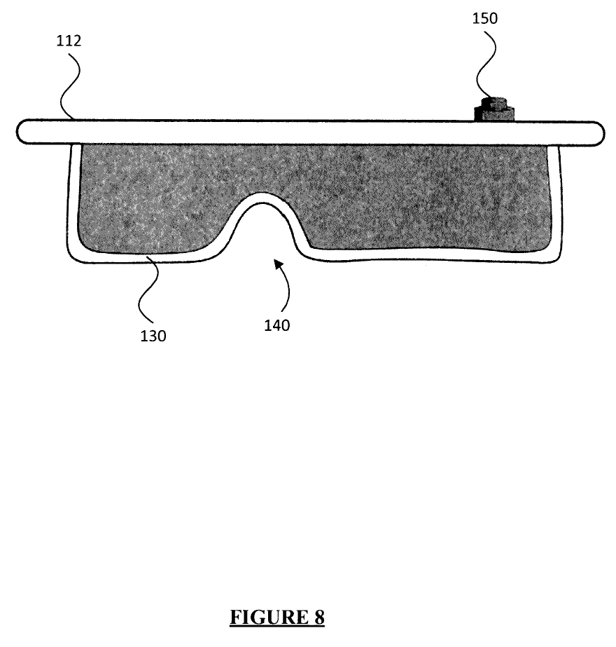

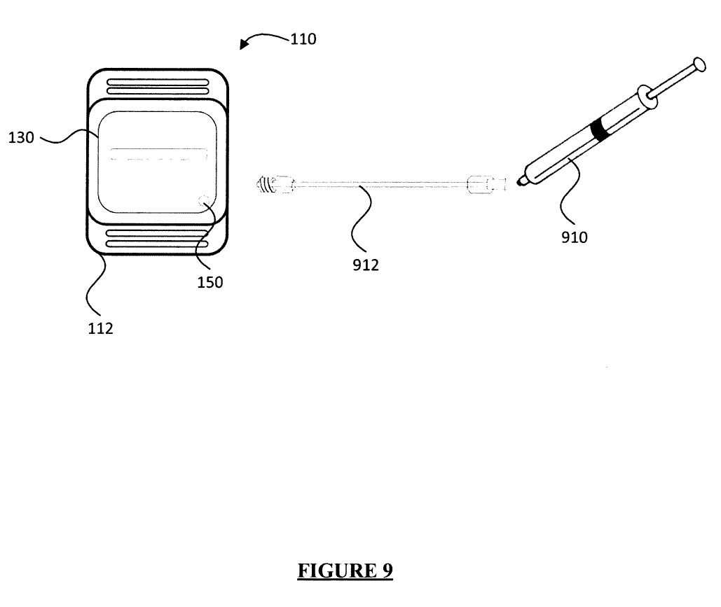

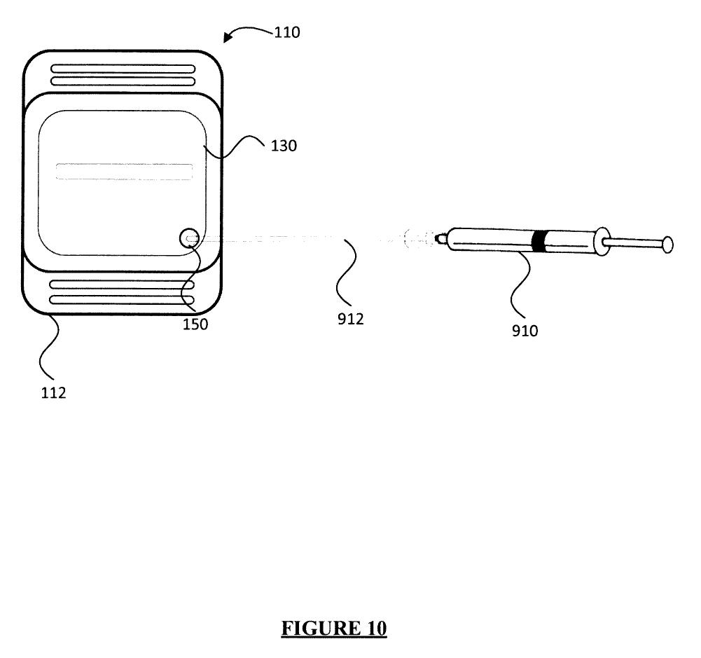

Recess 140 may be of various shapes as well. For instance, at the bottom end of recess 140 may be rounded, squared, or come to a point. As illustrated in FIG. 4-5, recess 140 is rounded at the bottom. The manner in which recess 140 is formed may depend on the particular incision that is being covered, or alternatively the shape of recess 140 may depend on cost-efficiency in terms of manufacturing. In addition, housing 130 may include multiple recesses if necessary. For example, multiple recesses may be used if the particular site of the body includes multiple incisions or wounds where reduced pressure is desirable. In addition, although recess 140 as depicted in the figures extends across the entire balloon, recess 140 may cover only a fraction of housing 130. For instance, recess 140 may be a slit in housing 130 and not extend to the ends housing 130. As another example, a lump of housing 130 may be removed from a center of housing 130, and the end portions of housing 130 may all be in tact.

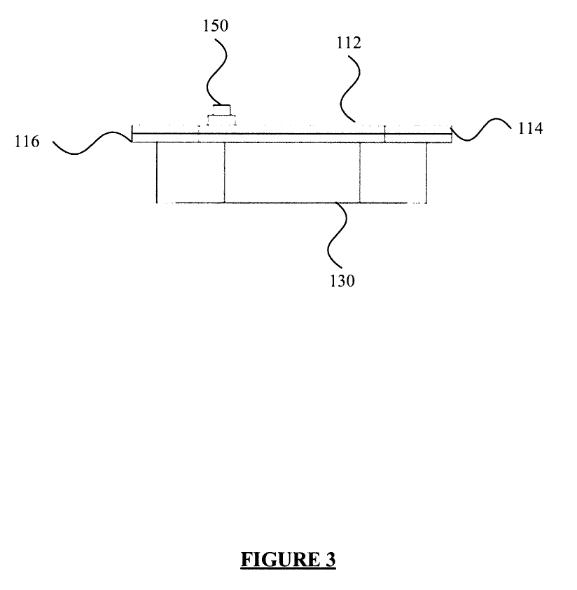

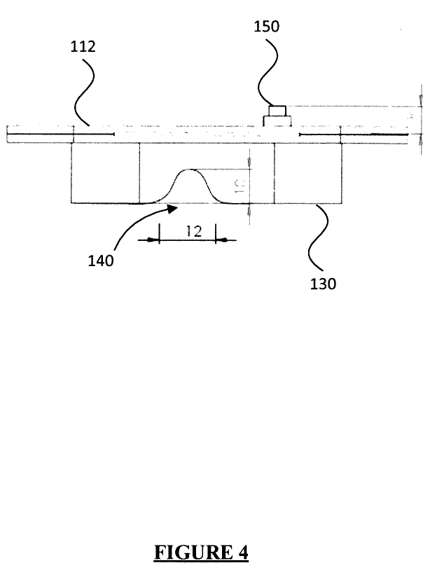

Housing 130 may include a valve 150 to empty and fill housing 130 with a gas or liquid. As shown in FIGS. 1 and 2, valve 150 is positioned on a top portion of housing 130. In this regard, valve 150 allows a person to extract any air or fluid that is located in housing 130. In addition, valve 150 allows the person to input air or fluid into housing 130. As one example, if the person wants to put a particular type of fluid in housing 130, the person may extract a first fluid and then input a second fluid. For instance, the person may want to exchange water with a saline solution. Housing 130 may be capable of housing and switching between multiple types of materials, such as switching between gas, liquid, and solids. For instance, if liquid is placed in housing 130, then the person may place housing 130 or the entire compression device 110 into a freezer, thereby freezing the contents of the housing 130 and turning the contents into a solid. The temperature and materials in housing 130 may depend on the particular type of incision, wound, or other infirmity.

A syringe 910 may be used to empty or fill housing 130. For instance, as shown in FIGS. 9 and 10, syringe 910 is configured to attach to line 912, line 912 in turn connects to valve 150 of compression device 110. The user is able to fill and empty housing 130 by either injecting the contents of syringe 910 into housing 130 or drawing the contents of housing 130 into syringe 910. Line 912 may act as a sealing mechanism in order to have an air-tight connection among valve 150, line 912, and syringe 910. It should be noted that in the example shown a male-female luer lock line and a male luer lock syringe is implemented; however, any line or syringe may be used as well. In addition, line 912 may not be necessary, but rather syringe 910 and valve 150 may only be used to inject and extra contents into and out of housing 130, respectively.

Compression device 110 may include a strap that is attached to frame 112 and secures compression device 110 to a body of the user. For instance, as shown in FIG. 1, frame 112 includes openings 170 and 172 that strap 160 inserts into. Strap 160 may include a buckle or other mechanism to adjust the tightness (e.g., tighten, loosen) of compression device 110 to the body. It should be noted that although the openings 170 and 172 are depicted as being cylindrical, there is no particular shape or size requirement of openings 170 and 172. In addition, as shown in FIGS. 11A-C, strap 160 may have one part that comes across one side of the body and another part of strap 160 may come across a second side of the body to more tightly secure compression device 110 to the body. Alternatively, there may be only a single part of the strap that comes across the body, or alternatively there may be multiple parts of the strap for greater adjustability and precision.

The compression device disclosed herein is advantageous because it allows for external cutaneous compression of an incision after the implantation of a device in a person's heart. For example, the compression device may be used after the implantation of a pace maker or defibrillator, the compression device includes a recess thereon that relieves any direct compression on the surgical site incision. In this regard, the compression device reduces the possibility of hematoma formation post-sub-muscular or subcutaneous device implantation. Although the compression device is disclosed herein as being applied to an incision post cardiac device implantation, the compression device may be used for other incisions and wounds as well where it is desirable to apply compression while simultaneously reliving direct compression directly on the surgical site or wound.

In addition, complications that result from pocket hematoma as a result of post device implantation can be costly to the healthcare system and/or increase a patient's in-hospital mortality. By reducing the possibility of post surgery injuries (e.g., hematoma) or other injuries that result from an incision or wound be utilizing the compression device disclosed herein, then not only will patient's obtain a benefit with respect to their well-being, but the overall healthcare system will see a benefit as well.

Most of the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

1. A compression device, comprising:
a frame including a first opening;
a strap secured through the first opening, wherein the strap is configured to secure the compression device to a body of a user; and
a housing that is attached to the frame, the housing having a bottom side and a top side, wherein the bottom side is configured to rest against a body of a user and the top side is opposite the bottom side, the bottom side of the housing having a recess that extends from a first end of the housing to a second end of the housing, wherein the first and second ends are opposite each other.

2. The compression device of claim 1, wherein the housing is removably attachable to the frame.

3. The compression device of claim 2, wherein multiple types of housings are attachable to the frame.

4. The compression device of claim 2, wherein the housing is removably attachable to the frame via a press fit or friction fit.

5. The compression device of claim 1, wherein the housing includes multiple recesses thereon.

6. The compression device of claim 1, wherein the housing is filled with a liquid or a solid.

7. The compression device of claim 6, wherein the liquid includes a saline solution.

8. The compression device of claim 1, further comprising a valve positioned on the top side of the housing, wherein the valve permits the removal and addition of gas or liquid into the housing.

9. The compression device of claim 1, wherein the recess is off-centered, wherein the off-centered recess is configured to cover an incision or wound based on the positioning of the incision or wound on the body of the user.

10. A kit comprising:
a frame including a first opening;
a strap secured through the first opening, wherein the strap is configured to secure compression device to a body of a user; and
a first housing that is attachable to the frame, the first housing having a bottom side and a top side, wherein the bottom side is configured to rest against a body of a user and the top side is opposite the bottom side, the bottom side of the first housing having a first recess that extends from a first end of the first housing to a second end of the first housing, wherein the first and second ends are opposite each other, the first housing including a gas, liquid, or solid material therein.

11. The kit of claim 1, further comprising:
a second housing having a second recess that is attachable to the frame, wherein the second housing includes a different gas, liquid, or solid material therein from the first housing.

12. The kit of claim 11, wherein the first and second housings are removably attachable to the frame via a press fit or friction fit.

13. The kit of claim 10, wherein the first housing includes multiple recesses thereon.

14. The kit of claim 10, wherein the first housing stores a saline solution.

15. The kit of claim 10, further comprising a valve positioned on the top side of the first housing, wherein the valve permits the removal and addition of the gas or liquid into the housing.

16. The kit of claim 10, wherein the recess is off-centered and the off-centered recess is configured to cover an incision or wound based on the positioning of the incision or wound on the body of the user.
